(19) United States
(12) Patent Application Publication
(10) Pub. No.: US 2012/0258183 A1
(43) Pub. Date: Oct. 11, 2012

(54) MACRONUTRIENT SENSITIVITY

(76) Inventors: Graeme John Smith, Victoria (AU); Nick Argyrou, Victoria (AU); Helen Argyrou, Victoria (AU); Harry Banaharis, Prahram (AU)

(21) Appl. No.: 13/501,867

(22) PCT Filed: Oct. 18, 2010
(86) PCT No.: PCT/AU2010/001384

§ 371 Date: Jun. 29, 2012

(30) Foreign Application Priority Data
Oct. 16, 2009 (AU) 2009905061

(51) Int. Cl. C12Q 1/68; C40B 40/06; A23L 1/00; A61P 3/08; A61P 3/06; C40B 30/00; A61K 36/71

(52) U.S. Cl. 424/726; 435/6.11; 506/7; 506/16; 426/2

(57) ABSTRACT

The present invention relates to a method and a kit for identifying a subjects macronutrient sensitivity. The method involves assaying a genetic sample from the subject to determine a polymorphism profile, analysing the polymorphism profile to identify risk alleles and determining the macronutrient sensitivity based on the number of risk alleles present. This information can be used for determining an appropriate diet to induce satiety, formulating a diet for inducing satiety, or for treating a range of medical complaints associated with metabolism.

### FIELD

The invention relates to a method for identifying a macronutrient sensitivity of a subject. The invention also relates to a method for formulating a diet for inducing satiety and a method for determining satiety in a subject. Furthermore, the invention relates to a kit suitable for use in the methods of the invention.

### BACKGROUND

Food is composed of three macronutrients and numerous micronutrients. The three macronutrients are carbohydrate, lipid and protein, whereas the micronutrients comprise a variety of compounds including trace minerals and vitamins.

Anthropological studies have suggested that an evolutionary adaptation to a specific food type may be behind the different responses to diet between individuals. In some parts of the world the ancient natural diet may have been more meat-based and individuals descended from such groups may be more suited to a high-protein, low-carbohydrate diet. In other parts of the world the ancient natural diet may have been more plant-based or grain-based and individuals descended from such origins may be more suited to a high-carbohydrate, low-fat diet.

It has been proposed also that a scarcity of a particular macronutrient in the ancient natural diet may have led to genetic adaptations that enable macronutrient metabolite turnover to be altered in order to retain systemically more of that macronutrient. Accordingly, it has been postulated that the body evolved over time to treat the scarce macronutrient as precious and to harvest as much of it as possible whenever it was available.

The modern Western diet provides unlimited access to all of the macronutrients and thus these ancient adaptations are no longer required. In fact, since the human body has not evolved to cope with such abundance of all of the macronutrients, such adaptations can be detrimental to an individual.

Most people adhering to a Western diet consume a similar macronutrient profile. Despite this, there are highly varied responses to such a diet, with systemic accumulation of particular macronutrients leading to pathological consequences in some individuals and not in others. Some of the pathologies associated with inappropriate macronutrient accumulation are obesity, insulin resistance, leptin resistance, type II diabetes and sugar addiction, and complications associated with each.

Historically, diets designed for weight loss and/or health improvement have been based largely on actively enforced caloric restriction, or caloric restriction combined with lipid reduction and carbohydrate increase. These diets have been largely unsuccessful in addressing the problem of weight loss and/or reduction in overall body fat as they are extremely difficult for the subject to maintain.

Weight loss, and maintenance of weight loss over time, can differ substantially between individuals. It has been suggested that this difference may result from differences between individuals at the genetic level.

There is a clinically established genetic relationship between obesity and metabolic disorders. This relationship may be caused by single-gene or multi-gene patterns of inheritance.

Previous methods for the diagnosis and/or treatment of metabolic disorders linked to genetic polymorphisms or genotypes have focused on analysing a single gene putatively involved with the regulation of metabolism to determine whether an individual is susceptible to increased appetite. However, previous methods have failed to account for satiety, which is the physiological feedback mechanism suppressing appetite. Therefore, a need exists for an alternative or improved method for the diagnosis and/or treatment of a metabolic disorder linked to a genetic polymorphism, specifically accounting for satiety.

It is to be understood that if any prior art publication is referred to herein such reference does not constitute an admission that the publication forms a part of the common general knowledge in the art in Australia or any other country.

### SUMMARY

A first aspect provides a method for identifying a subject's macronutrient sensitivity, comprising the steps of assaying a genetic sample from the subject for a polymorphism in a gene selected from the group consisting of TCF7L2(1), TCF7L2(2), KIR6.2(KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2) APOE, APOB(1), APOB(2), PSRC1, LDLR, CETP(1), CETP(2), LPL(1), LPL(2), PCSK9, FABP2, LEPR(1) and LEPR(2) or combination thereof, to determine a polymorphism profile, analysing said polymorphism profile to identify risk alleles and determining the macronutrient sensitivity of said subject based on the number of risk alleles present.

The identification of a subject's macronutrient sensitivity allows the provision of a diet plan taking into account this macronutrient sensitivity to allow the subject to achieve optimal satiety for initiating and maintaining weight loss, reducing body fat, ameliorating metabolic syndrome, improving health and well being, and managing food intolerance, for example.

The method may provide an integrated approach to satiety by accounting for both the genetic profile of the subject and the most appropriate macronutrient composition for the subject that will respond to the subject's genetic profile.

In one embodiment, the method comprises assaying a genetic sample from the subject for at least one polymorphism in each of 2, 3, 4, 5, 6, 7, 8, 9, 10, 11, 12, 13, 14, 15, 16 or 17 genes selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1) and APOA5(2).

In another embodiment, the method comprises assaying a genetic sample from the subject for at least one polymorphism in each of 2, 3, 4, 5, 6, 7, 8, 9, 10, 11, 12 or 13 genes selected from the group consisting of APOE, APOB(1), APOB(2), PSRC1, LDLR, CETP(1), CETP(2), LPL (1), LPL(2), PCSK9, FABP2, LEPR(1), and LEPR(2).

In yet another embodiment, the method comprises assaying a genetic sample from the subject for at least one polymorphism in each of 2, 3, 4, 5, 6, 7, 8, 9, 10, 11, 12, 13, 14, 15, 16, 17, 18, 19, 20, 21, 22, 23, 24, 25, 26, 27, 28, 29 or 30 genes selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2), APOE, APOB(1), APOB(2), PSRC1, LDLR, CETP(1), CETP(2), LPL (1), LPL(2), PCSK9, FABP2, LEPR(1), and LEPR(2).

The polymorphism may be a single nucleotide polymorphism (SNP).

The method may comprise the step of assaying the genetic sample to determine a haplogroup. The step of assaying the genetic sample to determine a haplogroup may comprise assaying a mitochondrial polymorphism or a Y-chromosome polymorphism.

In one embodiment, the method comprises the step of calculating a score from the polymorphism profile. The method may also comprise the step of determining the macronutrient sensitivity based on the score.

The macronutrient sensitivity identified by the method can be non-sensitive, carbohydrate sensitive, lipid sensitive or carbohydrate and lipid sensitive.

In a particular embodiment, the genetic sample of the method is a buccal sample.

In another embodiment, the method comprises the step of formulating a diet for the subject based on their macronutrient sensitivity. Formulating the diet may comprise prescribing the diet or providing the diet as food.

According to one embodiment of the method, the diet comprises a meal replacement food or supplement. The diet may comprise a liquid food, such as a long-life liquid food, a solid food, such as a bar or a powder, or any other edible item designed to be a meal replacement. The liquid food may be a shake.

In order to enhance the benefit of knowing one's macronutrient sensitivity or to enhance the effect of observing a diet prescribed on the basis of that macronutrient sensitivity, the method may be combined with counselling and/or exercise and may be supervised by a qualified healthcare professional. Counselling is chiefly aimed at improving a subject's knowledge regarding healthy lifestyle habits and factors that contribute to weight gain as well as to provide support and guidance to implement healthy changes, whereas exercise is mainly aimed at improving the physical well-being of a subject. The mental well-being of a subject encompasses their education and support. Thus, the method contemplates a holistic approach to satiety, where the benefit of observance of a macronutrient sensitivity or compliance with a formulated diet can be enhanced by supplementary activities.

In another embodiment, the method comprises the step of counselling the subject. Additionally, the method may comprise the step of providing an exercise regimen the subject. The exercise regimen may comprise aerobic exercise or anaerobic exercise.

In a particular embodiment, the method comprises the step of administering to the subject a nutraceutical or pharmaceutical substance. The nutraceutical may aid in normalising circulating glucose levels or circulating lipid and/or triglyceride levels.

A second aspect of the invention provides a method for determining an appropriate diet to induce satiety in a subject, comprising the steps of identifying the subject's macronutrient sensitivity by the method of the first aspect.

A third aspect of the invention provides a method for formulating a diet for inducing satiety in a subject, comprising the steps of assaying a genetic sample from the subject for a polymorphism in a gene selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2), APOE, APOB(1), APOB(2), PSRC1, LDLR, CETP(1), CETP(2), LPL (1), LPL(2), PCSK9, FABP2, LEPR(1), and LEPR(2) or combination thereof, to determine a polymorphism profile, and formulating a diet based on that polymorphism profile.

A fourth aspect of the invention provides a kit, comprising a genetic sampler for obtaining a genetic sample from a subject, when the genetic sample is assayed according to the method of the first aspect.

A fifth aspect of the invention provides a kit for identifying a macronutrient sensitivity of a subject, comprising a reagent for assaying a genetic sample obtained from the subject for a polymorphism in a gene selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2), APOE, APOB(1), APOB(2), PSRC1, LDLR, CETP(1), CETP(2), LPL (1), LPL(2), PCSK9, FABP2, LEPR(1), and LEPR(2) or combination thereof.

### DETAILED DESCRIPTION

Analysis of a subject's genetic profile, with regard to satiety polymorphisms, provides information that can be used to select a diet comprising appropriate ratios of satiety-inducing macronutrients and the foods that contain them for the individual's profile and this should lead to weight loss and/or body fat reduction without having to actively enforce reduced caloric intake or endure increased sensation of hunger.

Whilst a subject may consider that they are aware of their “trigger” foods for weight gain, for example, the present method provides a systematic approach, with scientific validation, to identifying specific foods or types of foods that should be avoided. Moreover, the present method enables those foods to be substituted with more appropriate foods for any individual. Indeed, the diet may be formulated to adjust the composition or ratio of one or more of the macronutrients in a personalised manner.

The methods disclosed can be used for induction of satiety and for determining a beneficial, ideally optimal, dietary macronutrient composition for inducing satiety in an individual, based on analysis of an individual's genetic profile with regard to a genotype known to be associated with the regulation of metabolism.

Different macronutrients exhibit different satiation responses in different people, with protein generally having the most lasting satiation effect. In one example, carbohydrate generally induces the least satiety in people of Caucasian origin relative to other groups.

While not wishing to be bound to any particular hypothesis, it has been postulated that in human ancestors, for example, the greater the abundance of a particular macronutrient, the greater the satiety response provided by that macronutrient. Apparently, this is because the macronutrient was readily available and did not need to be stored by the body. Since protein formed a large part of the ancient natural diet, this macronutrient was not regarded by the body as precious causing a higher level of satiety than carbohydrate, which was relatively scarce.

People who are descended from populations adapted to a high-protein, low-carbohydrate diet have a tendency to become overweight when they eat a diet high in carbohydrate, since they do not experience appropriate satiety responses in the absence of adequate amounts of protein. Such people would be deemed carbohydrate sensitive and with modern diets would have metabolic issues related to the processing of carbohydrates that would increase the risk of developing diseases such as type 2 diabetes. Conversely, people who are descended from populations adapted to a high-carbohydrate, low-fat diet have a tendency to become overweight when they eat a diet high in fat in the absence of adequate amounts of carbohydrate.

In addition to these basic responses, refined carbohydrates are in evolutionary terms a very recent addition to the human diet and there has not been sufficient time for genetic adaptation to this new type of food or to its abundance.

In short, a subject's genetically-determined macronutrient sensitivity is considered to be proportional to the subject's ancestrally-derived requirement for macronutrients of low abundance.

The hypothalamus is responsible for certain metabolic processes, in particular appetite. It synthesizes and secretes neurohormones, often called hypothalamic-releasing hormones, and these in turn stimulate or inhibit the secretion of pituitary hormones. It has been established that a reduction in refined carbohydrates combined with the introduction of protein can re-establish appropriate hypothalamic control of appetite.

In addition to controlling appetite and other metabolic processes, the hypothalamus also regulates satiety. Previous methods for the diagnosis and/or treatment of metabolic disorders linked to genetic polymorphisms or genotypes have focused on analysing single genes potentially involved with the regulation of metabolism. Moreover, these previous methods designed for weight loss and/or reduction in overall body fat have focused on appetite and have failed to address satiety. These previous methods have been largely unsuccessful because they are extremely difficult for the subject to maintain due to a lack of satiety. This lack of satiety is linked to a high degree of relapse into unhealthy eating habits. However, it has been established that introduction of a high-satiety macronutrient such as protein can re-establish appropriate hypothalamic control of appetite and satiety.

It is important to note the difference between appetite, which is the physiological drive to consume food, and satiety, which is the feedback mechanism by which the body signals that sufficient food has been consumed to satisfy the body's immediate energy requirements. The present disclosure does not identify genetic susceptibilities to increased appetite; rather it identifies genetic predisposition and a beneficial macronutrient composition required to induce satiety.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It must also be noted that, as used in the subject specification, the singular forms “a”, “an” and “the” include plural aspects unless the context clearly dictates otherwise.

“Appetite” as used herein refers to the physiological drive to consume food. Appetite is driven by the need for energy and nutrients by the body of the subject. Satiety represses appetite.

“Satiety” as used herein refers to the physiological feedback mechanism by which the body of the subject signals that sufficient food has been consumed to satisfy the subject's immediate energy requirements.

The term “inducing satiety” has its ordinary meaning, i.e. to bring about, produce, or cause satiety. The term is used in a relative sense such that satiety is induced with respect to satiety that may exist in the absence of the method or used disclosed herein. That is, satiety induced by the method or use of this disclosure has a greater magnitude than satiety that may exist otherwise.

The “subject” includes a mammal. The mammal may be a human, or may be a domestic, zoo, or companion animal. While it is particularly contemplated that the method and uses disclosed herein are suitable for humans, they are also applicable to animals, including treatment of companion animals such as dogs and cats, and domestic animals such as horses, cattle and sheep, or zoo animals such as felids, canids, bovids, and ungulates. In one embodiment, the subject is a human. The term “subject” is used interchangeably with “individual” and “person”.

“Consume” as used herein means to ingest by eating, drinking or otherwise introducing into the body some form of nutrient and may be used interchangeably with the term “feed” or “eat”.

The term “genotype” refers to the fundamental biochemical composition of the genetic material of an individual organism, and implicitly refers to the differences in that composition between individuals. Accordingly, the term “genotyping” refers to the act of assaying to determine the composition of the genetic material of an individual organism, often for comparison to the genotype of another individual. A genotype is usually determined from a polymorphism.

A “polymorphism” refers to the existence of two or more forms or variations in the DNA of a particular gene that has a frequency of at least 1% in the population. In the context of a genotype, it refers to the existence of two or more forms of a genotype, which differ in their nucleotide composition. A polymorphism includes a restriction fragment length polymorphism (RFLP), a tandem repeat, a variable number tandem repeat (VNTR), a short tandem repeat (STR), a minisatellite, a microsatellite, a simple sequence length polymorphism (SSLP), in insertion-deletion (indel), an amplified fragment length polymorphism (AFLP), a random amplification of polymorphic DNA (RAPD), a single nucleotide polymorphism (SNP), and any other genetic feature that may be distinguished between individuals. In one embodiment, the polymorphism is a SNP. Polymorphisms exist in at least two states or alleles.

As used herein, “polymorphism profile” refers to the combination of polymorphisms possessed by an individual with regard to the parts of the genome assessed. An individual's polymorphism profile, comprising one or more genotypes, can be used to differentiate between individuals who are likely to exhibit different responses to a particular stimulus, in this instance, to satiety.

In some embodiments, the polymorphism profile is used to calculate a score that indicates the likelihood that an individual will be sensitive to the macronutrient that is linked to the polymorphism assessed.

Similarly, “genetic profile” as used herein refers to the combination of alleles possessed by an individual with regard to the parts of the genome assessed.

In one embodiment, the method comprises assaying at least one polymorphism in each of 2, 3, 4, 5, 6, 7, 8, 9, 10, 11, 12, 13, 14, 15, 16 or 17 genes selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1) and APOA5(2).

In another embodiment, the method comprises assaying at least one polymorphism in each of 2, 3, 4, 5, 6, 7, 8, 9, 10, 11, 12 or 13 genes selected from the group consisting of APOE, APOB(1), APOB(2), PSRC1, LDLR, CETP(1), CETP(2), LPL (1), LPL(2), PCSK9, FABP2, LEPR(1), and LEPR(2).

In yet another embodiment, the method comprises assaying for at least one polymorphism in each of 2, 3, 4, 5, 6, 7, 8, 9, 10, 11, 12, 13, 14, 15, 16, 17, 18, 19, 20, 21, 22, 23, 24, 25, 26, 27, 28, 29 or 30 genes selected from the group consisting of TCF7L2(1), TCF7L2(2), KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2), APOE, APOB(1), APOB(2), PSRC1, LDLR, CETP(1), CETP(2), LPL (1), LPL(2), PCSK9, FABP2, LEPR(1), and LEPR(2).

“Allele” as used herein refers to one of the two copies of a genetic unit contained within an individual's genome. In a population, more then two alleles may exist. However, any individual will usually only possess a subset of alleles present in the population. For example, a mammalian individual will possess two alleles for a particular gene, although the population may comprise three or more alleles.

A “risk allele” refers to the specific allele of a genotype that confers a higher probability of sensitivity to a particular macronutrient.

“Single nucleotide polymorphism” or “SNP” as used herein means an alteration of a single nucleotide at a defined position within the genome of at least two individuals of the same species. SNPs usually comprise two alternative nucleotides, for example A or T, or, C or G. Such a SNP can be used to predict an individual's satiety response to the consumption of a particular macronutrient.

Two panels of SNPs, of which any one or more SNP may be genotyped, have been developed for determining the likelihood that a person will suffer reduced satiety and adverse metabolic effects from consuming carbohydrate or lipid in excess of the optimal level dictated by their genotype.

The first panel indicates the likelihood that a person will suffer reduced satiety and adverse metabolic effects from consuming excess carbohydrate. This panel is referred to as the carbohydrate sensitive panel and comprises the following SNPs:

TCF7L2(1) (encoding transcription factor 7-like 2 (T-cell specific, HMG-box)) NCBI unique identifier RS12255372 which is located on chromosome 10 of the Homo sapiens genome and comprises the following sequence (SEQ ID NO: 1):

TGCCCAGGAATATCCAGGCAAGAAT[G/T]ACCATATTCTGATAATTAC
TCAGGC

where the risk allele is the T genotype.
TCF7L2(2) (encoding transcription factor 7-like 2 (T-cell specific, HMG-box)) NCBI unique identifier RS7903146 which is located on chromosome 10 of the Homo sapiens genome and comprises the following sequence (SEQ ID NO: 2):

TTAGAGAGCTAAGCACTTTTTAGATA[C/T]TATATAATTTAATTGCCG
TATGAGG

where the risk allele is the T genotype.
KIR6.2 (KCNJ11) (encoding potassium inwardly-rectifying channel, subfamily J, Member 11; ATP-binding cassette sub-family C (CFTR/MRP) member 8) NCBI unique identifier RS5219 which is located on chromosome 11 of the Homo sapiens genome and comprises the following sequence (SEQ ID NO: 3):

CCGCTGGCGGGCACGGTACCTGGGCT[C/T]GGCAGGGTCCTCTGCCAG
GCGTGTC

where the risk allele is the T genotype.
PPARG (encoding peroxisome proliferator-activated receptor gamma) NCBI unique identifier RS1801282 which is located on chromosome 3 of the Homo sapiens genome and comprises the following sequence (SEQ ID NO: 4):

AAACTCTGGGAGATTCTCCTATTGAC[C/G]CAGAAAGCGATTCCTTCA
CTGATAC

where the risk allele is the C genotype.
IGFBP2 (encoding insulin-like growth factor 2 mRNA binding protein 2) NCBI unique identifier RS4402960 which is located on chromosome 3 of the Homo sapiens genome and comprises the following sequence (SEQ ID NO: 5):

CAGTAAGGTAGGATGGACAGTAGATT[G/T]AAGATACTGATTGTGTTT
GCAAACA

where the risk allele is the T genotype.
CDKN2B (encoding cyclin-dependent kinase inhibitor 2B) NCBI unique identifier RS10811661 which is located on chromosome 9 of the Homo sapiens genome and comprises the following sequence (SEQ ID NO: 6):

GCAGCTCACCTCCAGCTTTAGTTTTC[C/T]CATGACAGTAAGTCTATT
ACCCTCC

where the risk allele is the T genotype.
FTO (encoding fat mass and obesity associated protein) NCBI unique identifier RS9939609 which is located on chromosome 16 of the Homo sapiens genome and comprises the following sequence (SEQ ID NO: 7):

AGGTTCCTTGCGACTGCTGTGAATTT[A/T]GTGATGCACTTGGATAGT
CTCTGTT

where the risk allele is the A genotype.
SLC30A8 (encoding solute carrier family 30 (zinc transporter), member 8) NCBI unique identifier RS13266634 which is located on chromosome 8 of the Homo sapiens genome and comprises the following sequence (SEQ ID NO: 8):

GTGCTTCTTTATCAACAGCAGCCAGC[C/T]GGGACAGCCAAGTGGTTC
GGAGAGA

where the risk allele is the C genotype.
HHEX (encoding hematopoietically expressed homeobox) NCBI unique identifier RS1111875 which is located on chromosome 10 of the Homo sapiens genome and comprises the following sequence (SEQ ID NO: 9):

CTCCGTACCATCAAGTCATTTCCTCT[A/G]GACGTCTGAACCTGCACT
CAGGGTC

where the risk allele is the G genotype.
CDKAL1 (encoding CDK5 regulatory subunit associated protein 1-like 1) NCBI unique identifier RS7756992 which is located on chromosome 6 of the Homo sapiens genome and comprises the following sequence (SEQ ID NO: 10):

AATATTCCCCCCTGTATTTTAGTTTT[A/G]GATCTACAGTTATGTAGC
AATGAGC

where the risk allele is the G genotype.
WFS1 (encoding Wolfram syndrome 1 (wolframin)) NCBI unique identifier RS10010131 which is located on chromosome 4 of the Homo sapiens genome and comprises the following sequence (SEQ ID NO: 11):

GCACACAAGGCCTTTGACCACATCCT[A/G]TCCCTCAGGCATCACGTC
CGAGAAC

where the risk allele is the G genotype.
NOTCH2 (encoding Notch homolog 2) NCBI unique identifier RS10923931 which is located on chromosome 1 of the Homo sapiens genome and comprises the following sequence (SEQ ID NO: 12):

TCTTGTTGCTCCATCCTCTGGCTTCA[G/T]GCTGAACAAGTAAGATTA
TGGGCAC

where the risk allele is the T genotype.
JAZF1 (encoding JAZF zinc finger 1) NCBI unique identifier RS864745 which is located on chromosome 7 of the Homo sapiens genome and comprises the following sequence (SEQ ID NO: 13):

CATTTCCTACAACCATTCAAAACATT[A/G]TAACAGTTCAAATTATAT
TTGAGCA

where the risk allele is the A genotype.
CDC123 (encoding cell division cycle 123 homolog) NCBI unique identifier RS12779790 which is located on chromosome 10 of the Homo sapiens genome and comprises the following sequence (SEQ ID NO: 14):

ACCCGGACAATGTTGGGAATTTTTTC[A/G]TATTTCTTGGCCATTTAT
ATATCTT

where the risk allele is the G genotype.
G6PC2 (encoding glucose-6-phosphatase, catalytic, 2) NCBI unique identifier RS560887 which is located on chromosome 2 of the Homo sapiens genome and comprises the following sequence (SEQ ID NO: 15):

TCTACGATGGAAGAATAGATACAAGC[A/G]TAAAAAGCAAAGAAACTG
GATCACT

where the risk allele is the G genotype.
APOA5(1) (encoding apolipoprotein A-V) NCBI unique identifier RS12286037 which is located on chromosome 11 of the Homo sapiens genome and comprises the following sequence (SEQ ID NO: 16):

GACTATAGTACAATGTCTTTACCAAA[C/T]TGGAAGACCATAGTGCAG
TCTTCGA

where the risk allele is the T genotype.
APOA5(2) (encoding apolipoprotein A-V) NCBI unique identifier RS662799 which is located on chromosome 11 of the Homo sapiens genome and comprises the following sequence (SEQ ID NO: 17):

TGAGCCCCAGGAACTGGAGCGAAAGT[A/G]AGATTTGCCCCATGAGGA
AAAGCTG

where the risk allele is the G genotype.

The second panel of SNPs that has been developed indicates the likelihood that a person will suffer reduced satiety and adverse metabolic consequences from consuming excess lipid. This panel is referred to as the lipid sensitive panel and comprises the following SNPs:

APOE/APOC1 (encoding apolipoprotein E; apolipoprotein C-I) NCBI unique identifier RS4420638 which is located on chromosome 19 of the Homo sapiens genome and comprises the following sequence (SEQ ID NO: 18):

CAATGTCACTATGCTACACTTTTCCT[A/G]GTGTGGTCTACCCGAGAT
GAGGGGC

where the risk allele is the G genotype.
APOB(1) (encoding apolipoprotein B (including Ag(x) antigen)) NCBI unique identifier RS693 which is located on chromosome 2 of the Homo sapiens genome and comprises the following sequence (SEQ ID NO: 19):

CACATGAAGGCCAAATTCCGAGAGAC[C/T]CTAGAAGATACACGAGAC
CGAATGT

where the risk allele is the T genotype.
APOB(2) (encoding apolipoprotein B (including Ag(x) antigen)) NCBI unique identifier RS754523 which is located on chromosome 2 of the Homo sapiens genome and comprises the following sequence (SEQ ID NO: 20):

GTATTTGCAAAGTAGGTGACAATTGC[C/T]TAGTATCCCTAATATCAA
TACAAAA

where the risk allele is the C genotype.
PSRC1 (encoding proline/serine-rich coiled-coil 1) NCBI unique identifier RS599839 which is located on chromosome 1 of the Homo sapiens genome and comprises the following sequence (SEQ ID NO: 21):

AAAGAGAAAGAAATAGGAGCAGGATC[A/G]ACTTCCAGATATACAGAG
AATATAA

where the risk allele is the A genotype.
LDLR (encoding low density lipoprotein receptor) NCBI unique identifier RS6511720 which is located on chromosome 19 of the Homo sapiens genome and comprises the following sequence (SEQ ID NO: 22):

CTCACCAATCAACCTCTTCCTTAAGA[G/T]AAAATGTTAAGGAAGTCT
TAGGCAA

where the risk allele is the G genotype.
CETP(1) (encoding cholesteryl ester transfer protein, plasma) NCBI unique identifier RS5882 which is located on chromosome 16 of the Homo sapiens genome and comprises the following sequence (SEQ ID NO: 23):

TTGATTGGCAGAGCAGCTCCGAGTCC[A/G]TCCAGAGCTTCCTGCAGT
CAATGAT

where the risk allele is the A genotype.
CETP(2) (encoding cholesteryl ester transfer protein, plasma) NCBI unique identifier RS708272 which is located on chromosome 16 of the Homo sapiens genome and comprises the following sequence (SEQ ID NO: 24):

ACCTGGCTCAGATCTGAACCCTAACT[C/T]GAACCCCAGTGATTCTGG
GTCTCAG

where the risk allele is the C genotype.
LPL (1) (encoding lipoprotein lipase) NCBI unique identifier RS320 which is located on chromosome 8 of the Homo sapiens genome and comprises the following sequence (SEQ ID NO: 25):

ACAGAGATCGCTATAGGATTTAAAGC[G/T]TTTATACTAAATGTGCTG
GGATTTT

where the risk allele is the T genotype.
LPL (2) (encoding lipoprotein lipase) NCBI unique identifier RS328 which is located on chromosome 8 of the Homo sapiens genome and comprises the following sequence (SEQ ID NO: 26):

CCATGACAAGTCTCTGAATAAGAAGT[C/G]AGGCTGGTGAGCATTCTG
GGCTAAA

where the risk allele is the C genotype.
PCSK9 (encoding proprotein convertase subtilisin/kexin type 9) NCBI unique identifier RS11206510 which is located on chromosome 1 of the Homo sapiens genome and comprises the following sequence (SEQ ID NO: 27):

CAAGGATATAGGGAAAACCTTGAAAG[C/T]GATGTCTGTGGTGGCCGT
CTTTGGC

where the risk allele is the T genotype.
FABP2 (encoding fatty acid binding protein 2, intestinal) NCBI unique identifier RS1799883 which is located on chromosome 4 of the Homo sapiens genome and comprises the following sequence (SEQ ID NO: 28):

ATAAATTCACAGTCAAAGAATCAAGC[A/G]CTTTTCGAAACATTGAAG
TTGTTTT

where the risk allele is the A genotype.
LEPR (1) (encoding leptin receptor) NCBI unique identifier RS8179183 which is located on chromosome 1 of the Homo sapiens genome and comprises the following sequence (SEQ ID NO: 29):

ATAATTAATGGAGATACTATGAAAAA[C/G]GAGAAAAATGTCACTTTA
CTTTGGA

where the risk allele is the C genotype.
LEPR (2) (encoding leptin receptor) NCBI unique identifier RS1892534 which is located on chromosome 1 of the Homo sapiens genome and comprises the following sequence (SEQ ID NO: 30):

GGAACTTTGTGGTTGCAGTATGTCTT[A/G]ATCCATCAGCATATTGTC
CAACTCC

where the risk allele is the G genotype.

The assay may be performed against genes in one or both panels. If more than one gene is to be assayed for a polymorphism, the assays may be performed simultaneously or sequentially. If more than one gene is to be assayed for a polymorphism, the assays may be performed on distinct genetic samples from the same subject, for example spatially or temporally distinct samples.

In a certain embodiment, the SNP comprises SEQ ID NO: 1 (RS12255372), SEQ ID NO: 2 (RS7903146), SEQ ID NO: 3 (RS5219), SEQ ID NO: 4 (RS1801282), SEQ ID NO: 5 (RS4402960), SEQ ID NO: 6 (RS10811661), SEQ ID NO: 7 (RS9939609), SEQ ID NO: 8 (RS13266634), or SEQ ID NO: 9 (RS1111875), SEQ ID NO: 10 (RS7756992), SEQ ID NO: 11 (RS10010131), SEQ ID NO: 12 (RS10923931), SEQ ID NO: 13 (RS864745), SEQ ID NO: 14 (RS12779790), SEQ ID NO: 15 (RS560887), SEQ ID NO: 16 (RS12286037) or SEQ ID NO: 17 (RS662799). In another embodiment, The SNP comprises SEQ ID NO: 18 (RS4420638), SEQ ID NO: 19 (RS693), SEQ ID NO: 20 (RS754523), SEQ ID NO: 21 (RS599839), SEQ ID NO: 22 (RS6511720), SEQ ID NO: 23 (RS5882), or SEQ ID NO: 24 (RS708272), SEQ ID NO: 25 (RS320), SEQ ID NO: 26 (RS328), SEQ ID NO: 27 (RS11206510), SEQ ID NO: 28 (RS1799883), SEQ ID NO: 29 (RS8179183) or SEQ ID NO: 30 (RS1892534). In yet another embodiment the SNP comprises SEQ ID NO: 1 (RS12255372), SEQ ID NO: 2 (RS7903146), SEQ ID NO: 3 (RS5219), SEQ ID NO: 4 (RS1801282), SEQ ID NO: 5 (RS4402960), SEQ ID NO: 6 (RS10811661), SEQ ID NO: 7 (RS9939609), SEQ ID NO: 8 (RS13266634), or SEQ ID NO: 9 (RS1111875), SEQ ID NO: 10 (RS7756992), SEQ ID NO: 11 (RS10010131), SEQ ID NO: 12 (RS10923931), SEQ ID NO: 13 (RS864745), SEQ ID NO: 14 (RS12779790), SEQ ID NO: 15 (RS560887), SEQ ID NO: 16 (RS12286037), SEQ ID NO: 17 (RS662799), SEQ ID NO: 18 (RS4420638), SEQ ID NO: 19 (RS693), SEQ ID NO: 20 (RS754523), SEQ ID NO: 21 (RS599839), SEQ ID NO: 22 (RS6511720), SEQ ID NO: 23 (RS5882), or SEQ ID NO: 24 (RS708272), SEQ ID NO: 25 (RS320), SEQ ID NO: 26 (RS328), SEQ ID NO: 27 (RS11206510), SEQ ID NO: 28 (RS1799883), SEQ ID NO: 29 (RS8179183) or SEQ ID NO: 30 (RS1892534).

In one embodiment, the risk allele of SEQ ID NO: 1 (RS12255372) is T, SEQ ID NO: 2 (RS7903146) is T, SEQ ID NO: 3 (RS5219) is T, SEQ ID NO: 4 (RS1801282) is C, SEQ ID NO: 5 (RS4402960) is T, SEQ ID NO: 6 (RS10811661) is T, SEQ ID NO: 7 (RS9939609) is A, SEQ ID NO: 8 (RS13266634) is C, or SEQ ID NO: 9 (RS1111875) is G, SEQ ID NO: 10 (RS7756992) is G, SEQ ID NO: 11 (RS10010131) is G, SEQ ID NO: 12 (RS10923931) is T, SEQ ID NO: 13 (RS864745) is A, SEQ ID NO: 14 (RS12779790) is G, SEQ ID NO: 15 (RS560887) is G, SEQ ID NO: 16 (RS12286037) is T or SEQ ID NO: 17 (RS662799) is G.

In another embodiment, the risk allele of SEQ ID NO: 18 (RS4420638) is G, SEQ ID NO: 19 (RS693) is T, SEQ ID NO: 20 (RS754523) is C, SEQ ID NO: 21 (RS599839) is A, SEQ ID NO: 22 (RS6511720) is G, SEQ ID NO: 23 (RS5882) is A, or SEQ ID NO: 24 (RS708272) is C, SEQ ID NO: 25 (RS320) is T, SEQ ID NO: 26 (RS328) is C, SEQ ID NO: 27 (RS11206510) is T, SEQ ID NO: 28 (RS1799883) is A, SEQ ID NO: 29 (RS8179183) is C or SEQ ID NO: 30 (RS1892534) is G.

In yet another embodiment, the risk allele of SEQ ID NO: 1 (RS12255372) is T,

SEQ ID NO: 2 (RS7903146) is T, SEQ ID NO: 3 (RS5219) is T, SEQ ID NO: 4 (RS1801282) is C, SEQ ID NO: 5 (RS4402960) is T, SEQ ID NO: 6 (RS10811661) is T, SEQ ID NO: 7 (RS9939609) is A, SEQ ID NO: 8 (RS13266634) is C, or SEQ ID NO: 9 (RS1111875) is G, SEQ ID NO: 10 (RS7756992) is G, SEQ ID NO: 11 (RS10010131) is G, SEQ ID NO: 12 (RS10923931) is T, SEQ ID NO: 13 (RS864745) is A, SEQ ID NO: 14 (RS12779790) is G, SEQ ID NO: 15 (RS560887) is G, SEQ ID NO: 16 (RS12286037) is T, SEQ ID NO: 17 (RS662799) is G, SEQ ID NO: 18 (RS4420638) is G, SEQ ID NO: 19 (RS693) is T, SEQ ID NO: 20 (RS754523) is C, SEQ ID NO: 21 (RS599839) is A, SEQ ID NO: 22 (RS6511720) is G, SEQ ID NO: 23 (RS5882) is A, or SEQ ID NO: 24 (RS708272) is C, SEQ ID NO: 25 (RS320) is T, SEQ ID NO: 26 (RS328) is C, SEQ ID NO: 27 (RS11206510) is T, SEQ ID NO: 28 (RS1799883) is A, SEQ ID NO: 29 (RS8179183) is C or SEQ ID NO: 30 (RS1892534) is G.

The method of determining macronutrient sensitivity involves genotyping to identify the variation inherited at loci associated with macronutrient metabolism. For carbohydrate sensitivity, studies have shown loci associated with the genes including but not limited to TCF7L2 (rs12255372, rs7903146), KIR6.2 (KCJN11; rs5219), PPARG (rs1801282), IGF2BP2 (rs4402960), CDKN2B (rs10811661), FTO (rs9939609), SLC30A8 (rs13266634), HHEX (rs1111875), CDKAL1 (rs7756992), WFS1 (rs10010131), NOTCH2 (rs10923931), JAZF1 (rs864745), CDC123 (rs12779790), G6PC2 (rs560887) and APOA5 (rs12286037, rs662799).

For lipid sensitivity, studies have shown loci associated with the genes including but not limited to APOE (rs4420638), APOB (rs693, rs754523), PSRC1 (rs599839), LDLR (rs6511720), CETP (rs5882, rs708272), LPL (rs320, rs328), PCSK9 (rs11206510), FABP2 (rs1799883), and LEPR (rs8179183, rs1892534).

As the aim of genotyping is to identify if an individual is carrying gene versions that orient them to macronutrient sensitivity, polymorphisms for testing should be selected from both groups to determine the type of sensitivity. The greater the number of polymorphisms tested the greater the likelihood of identifying a genetic sensitivity associated with one or both macronutrients. Genotyping may be conducted by any means known in the art. For example, genotyping may include polymerase chain reaction (PCR), nucleic acid sequencing, primer extension reactions, or an array-based method.

In one embodiment, genotyping is performed using array or chip technology. A number of array technologies are known in the art and commercially available for use, including, but not limited to, static arrays (e.g. photolithographically set), suspended arrays (e.g. soluble arrays), and self assembling arrays (e.g. matrix ordered and deconvoluted).

Alternatively, a polymorphism can be detected in genetic material using techniques including direct analysis of isolated nucleic acids such as Southern blot hybridisation or direct nucleic acid sequencing. Another alternative for direct analysis of polymorphisms is the INVADER® assay (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample.

Conveniently, assaying a polymorphism may utilise genomic DNA. However, assaying a polymorphism may also be performed utilising mRNA or cDNA, for example. Assaying a polymorphism also encompassed indirectly assaying a genetic polymorphism by detecting a consequential difference in a gene product, for example, by detecting an amino acid substitution in cases where a polymorphism results in a codon change.

“Genome” or “genomic” as used herein refers to the complete genetic material encoding an organism.

As used herein, “gene” refers to any genetic material that provides instructions for the organism to perform some biological structure of function. Most commonly, but not exclusively, a gene will comprise one or more exons encoding the amino acid sequence of a polypeptide or protein, intervening introns, and non-coding regions including the promoter, 5′-untranslated region and the 3′-untranslated region. That is, a gene specifically included non-coding regions. The term “gene” also includes portions such as enhancer elements that may function in trans with the coding portion of a gene.

Because ancestry plays a role in genetic adaptation to diet, genotyping may include analysis of maternal and paternal haplogroups to further determine macronutrient sensitivity.

As used herein, a “haplotype” refers to a specific combination of alleles at two or more genetic loci that are transmitted together.

In turn, a “haplogroup”, is a collection of similar haplotypes and relates to genetic populations and ancestral origin. A haplogroup may be predicted from a haplotype. In one embodiment, a haplogroup comprises a mitochondrial polymorphism or haplogroup, which is maternal, or a Y-chromosome polymorphism or haplogroup, which is paternal.

A “genetic sample” comprises any form of genetic material specific to a subject. A genetic sample may be a deoxyribonucleic acid (DNA) or a ribonucleic acid (RNA), or any modification or derivative thereof. Thus, a genetic sample usually will include a cell derived from a subject. The genetic sample may be a blood sample, a mucosal sample, a saliva sample, a hair sample including a follicle, urine, mouth wash, amniotic fluid or other tissue or fluid sample that contains a cell, DNA or RNA that is suitable for genotyping. In one embodiment, the genetic sample is a buccal swab.

A genetic sample may be obtained using a “genetic sampler”, which refers to a device for obtaining DNA or RNA suitable for genotyping. A genetic sampler may be a swab, a scraper or a container or any device capable of capturing genetic material, such as a cell, for genotype analysis.

Genetic material may be isolated from the genetic sample by any method known in the art, for example extraction and precipitation or silica-based extraction.

A genetic sampler may be included in a kit. A kit may also include a reagent for detecting a genotype. For example, a reagent may include a support or support material such as, without limitation, a nylon or nitrocellulose membrane, bead, or plastic film, or glass, or microarray or nanoarray, comprising a set of polymorphisms from which a subject's macronutrient sensitivity may be determined. The kit may comprise other reagents necessary for performing the genotyping, including, but not limited to, labelled or unlabelled nucleic acid probes, detection label, buffers, and controls. The kit may include instructions for use.

In one embodiment, a kit comprises a reagent for assaying a genetic sample obtained from the subject for at least one polymorphism in each of 2, 3, 4, 5, 6, 7, 8, 9, 10, 11, 12, 13, 14, 15, 16, 17, 18, 19, 20, 21, 22, 23, 24, 25, 26, 27, 28, 29 or 30 genes selected from the group consisting of TCF7L2(1), TCF7L2(2) KIR6.2 (KCJN11), PPARG, IGF2BP2, CDKN2B, FTO, SLC30A8, HHEX, CDKAL1, WFS1, NOTCH2, JAZF1, CDC123, G6PC2, APOA5(1), APOA5(2), APOE, APOB(1), APOB(2), PSRC1, LDLR, CETP(1), CETP(2), LPL (1), LPL(2), PCSK9, FABP2, LEPR(1), and LEPR(2).

The kit would enable determination of whether a subject is genetically predisposed to macronutrient sensitivity. This information can be used to screen individuals, such as obese and overweight individuals, including children and adults and the elderly, and classify them based on their genetic predisposition for beneficial induction of satiety. The kit can also be used by individuals who have successfully lost weight, but who cannot maintain the weight loss, to determine if their difficulty in maintaining the weight loss is due to a genetic predisposition to sub-optimal satiety. Screening of normal weight individuals could help to identify people who possess a macronutrient sensitivity or are more likely to gain weight. Appropriate measures can then be implemented in diet, and possible lifestyle, medicinal and surgical interventions. Such a genetic approach will help professionals in the field of weight-management to improve targeting patients with appropriate advice regarding their weight management based on their macronutrient sensitivity.

“Diet” as used herein refers to the composition of nutrients that is consumed by an individual. Particularly envisaged is the composition of one or more macronutrients consumed by an individual. A “diet” may be a written or verbal prescriptive recitation of the composition of foods and/or nutrients for consumption. A “diet” also encompasses foods and/or nutrients in physical form for consumption.

The term “food” refers to a substance or material for consumption as a source of nutrients. A “food” may be comprised in a “diet”. In one embodiment, the food comprises one, two or three macronutrients in beneficial or optimal amounts or ratios. A food may be solid or liquid. A food may be dried, powdered, compressed, frozen, gelled or fresh, for example. A food may be in the form of a bar, a block, a biscuit, a crisp, a loaf, a spread, a paste, an emulsion, a suspension, a soup, a broth, a drink, a concentrate, a gel, or any other suitable form.

A “liquid food” refers to a substance or material for consumption as a source of nutrients in a liquid or flowable form. One example of a liquid food is a “shake”, which refers to any one of a number of liquid foods that may be shaken, blended, or otherwise combined. A “shake” often visually or texturally resembles a milkshake or thickshake. Other examples are a drink, a soup or a broth.

As used herein, the term “meal replacement” refers to a food that may be eaten or consumed alone to provide the composition of nutrients required by a subject, without any supplementary food items. A “meal replacement” may be prepared in advance in a ready-to-eat embodiment and provided to a subject, or may be prepared by the subject, for example by adding water to a dried, formulated food.

As used herein, the term “formulate” or “formulating” refers to the expression in precise form of the amount of a macronutrient in a diet. Alternatively, the ratio of a macronutrient relative to another dietary component may be stated in precise form. The formulation may be provided as a written or verbal prescriptive recitation on the selection of appropriate foods. Alternatively, the formulation may comprise provision of appropriate foods per se. In another embodiment, the formulation may be provided as a formulated food, for example a meal replacement formulation. In all cases, formulation provides adjustment for macronutrient composition or ratio according to the individual's requirements as determined by their genotype.

In one embodiment, the amount of one macronutrient or the ratio of one macronutrient to other dietary components is expressed when formulating a diet or food. In another embodiment, the amount of two macronutrients or the ratios of two macronutrients to other dietary components are expressed when formulating a diet or food. In another embodiment, the amount of three macronutrients or the ratios of three macronutrients to other dietary components are expressed when formulating a diet or food.

Dietary formulation based on the genetic profile of the subject and consumption of the formulated diet by the subject can induce innate hypothalamic-regulated satiety leading to weight loss without having to actively enforce reduced caloric intake or endure increased sensation of hunger. Furthermore, improvement of satiety should also control appetite by hypothalamic-regulated feedback inhibition.

“Macronutrient” as used herein refers to one of the major energy providing nutritional categories consisting of carbohydrate, protein or lipid. This is distinct from micronutrient, which refers to nutritional compounds that are not major sources of energy and are required in much smaller quantities. Examples of micronutrients include minerals and vitamins.

“Protein” is a class term referring to any protein or polypeptide composed of amino acids. Protein is a macronutrient and may be derived from animal source, vegetable source, or a combination of animal and vegetable sources.

“Carbohydrate” is a class term for simple organic compounds that are aldehydes or ketones with many hydroxyl groups added, usually one on each carbon atom that is not part of the aldehyde or ketone functional group. Carbohydrate is a macronutrient and is a common biological store of energy. Carbohydrate is generally obtained from vegetable sources, particularly grains and cereals. Carbohydrates can be classified as simple (monosaccharides and disaccharides) or complex (oligosaccharides and polysaccharides). A “refined carbohydrate” or “processed carbohydrate” refers to a grain source of carbohydrate in which processing has stripped the bran and germ from the whole grain.

“Lipid” is a class term referring to generally hydrophobic molecules, or amphiphilic molecules. Lipid may be derived from ketoacyl or isoprene groups. Lipid is a macronutrient and is a common biological store of energy. Lipid may be derived from animal source, vegetable source, or a combination of animal and vegetable sources. Lipid includes triacylglicerides (TAG, or triglycerides), phospholipids, fatty acids and sterols.

A “fatty acid” comprises a hydrocarbon chain and a terminal carboxylic acid group. Fatty acids may be divided into “saturated fatty acids”, comprising no unsaturated carbon-carbon double bonds in the hydrocarbon chain, and “unsaturated fatty acids”, comprising at least one carbon-carbon double bond in the hydrocarbon chain. A “monounsaturated fatty acid” comprises one carbon-carbon double bond in the hydrocarbon chain. A “polyunsaturated fatty acid” comprises at least two carbon-carbon double bonds in the hydrocarbon chain. Nutritionally important fatty acids include, for example, palmitic acid, stearic acid, oleic acid, linolenic acid, linoleic acid, arachidonic acid, eicosapentanoic acid and docosahexanoic acid.

A formulated diet or formulated food may comprise a nutritional supplement. Nutritional supplements include, for example, vitamins and minerals.

Vitamins that may be used as a nutritional supplement include vitamin A, biotin, vitamins B1, B2, B3, B5, B6, B12, folate, 5-methyltetrahydrofolate, vitamin C, vitamin D, vitamin E and vitamin K.

Minerals that may be used as a nutritional supplement include boron, calcium, chromium, chloride, copper, fluoride, iron, magnesium, manganese, molybdenum, potassium, phosphorus, sodium, selenium, vanadium, and zinc, including chemical complexes of these minerals.

A formulated diet or formulated food may comprise excipients, flavourings, colourings, sweeteners, and/or other ingredients to improve the effectiveness or sensory characteristics of the formulated diet or formulated food when consumed by the subject.

As used herein, “macronutrient sensitivity” refers to the physiological state of an individual who is genetically predisposed to reduced satiety after the consumption of foods comprising a particular macronutrient relative to the other macronutrients. This predisposition may be identified by the presence of one or more genotypes associated with metabolic function.

Individuals possessing one of the risk alleles from either of the genotype panels possess increased risk of reduced satiety and risk consequent metabolic disorders if they consume foods comprising macronutrient ratios that are incompatible with the respective macronutrient sensitivity group.

An individual can be classified as non-sensitive, carbohydrate sensitive, lipid sensitive or carbohydrate and lipid sensitive based on the number and type of risk alleles present in their genome.

For example, a subject possessing one risk allele from the carbohydrate sensitive panel may experience reduced satiety if they consume foods high in carbohydrate. Similarly, a subject possessing one risk allele from the lipid sensitive panel may experience reduced satiety if they consume foods high in lipid.

The more risk alleles from each respective panel that the individual possesses, the higher their risk is for reduced satiety and the development of a metabolic disorder.

The probability of having all polymorphisms in either the lipid sensitivity panel or the carbohydrate sensitivity panel is the multiplication of the frequency of the risk allele in the population, across all polymorphisms in the respective panels. For example, hypothetically—If the population frequency of the risk alleles A, B, and C was 10%, 15%, and 40% respectively then the probability of having risk alleles A, B & C is 10%×15%×40%, which is 0.6%.

In general, an individual who is homozygous for the susceptibility allele combination would have greater sensitivity to the relevant macronutrient than a person who is heterozygous for the susceptibility allele combination.

The carbohydrate and lipid sensitive classification refers to individuals that possess both carbohydrate and lipid sensitive risk alleles. For example, a subject possessing two or more risk alleles from one of these macronutrient sensitivity groups and two or more risk alleles from the other macronutrient sensitivity group would be classified carbohydrate and lipid sensitive.

“Carbohydrate sensitive” as used herein refers to the physiological state of an individual who is genetically predisposed to reduced satiety after the consumption of foods comprising excess carbohydrate relative to the other macronutrients and relative to their requirements. This predisposition may be identified by the presence of one or more genotypes associated with metabolic function. For a person identified as carbohydrate sensitive, a beneficial or optimal diet for inducing satiety will comprise a decreased amount of dietary macronutrient contribution from carbohydrate and/or from refined carbohydrate, and an increased amount of dietary macronutrient contribution from lipids.

“Lipid sensitive” as used herein refers to the physiological state of an individual who is genetically predisposed to reduced satiety after the consumption of foods comprising excess lipid relative to the other macronutrients and relative to their requirements. This predisposition may be identified by the presence of one or more genotypes associated with metabolic function. For a person identified as lipid sensitive, a beneficial or optimal diet for inducing satiety will comprise a decreased amount of dietary macronutrient contribution from lipid and/or from saturated lipid, and an increased amount of dietary macronutrient contribution from carbohydrate.

Further genetic analysis can be carried out on lipid sensitive individuals to determine whether dietary polyunsaturated or monounsaturated lipid is more beneficial for normalising blood triglyceride levels.

For a person identified as “carbohydrate and lipid sensitive”, the optimal diet for inducing satiety will comprise the least amount of dietary calories from carbohydrate and lipid sources, and the most dietary calories from protein sources.

Accordingly, a “macronutrient sensitivity group” comprises one or more individuals ascribed a particular macronutrient sensitivity.

As used herein, the term “score” refers to a numerical value calculated from the number of genotypes associated with a given macronutrient sensitivity possessed by a subject. A score may be modified based on the subject's haplotype, haplogroup or ancestral origin, for example as determined using a mitochondrial polymorphism or a Y-chromosome polymorphism.

Each gene polymorphism is selected based on its effect on altered lipid or carbohydrate metabolism. For example, the susceptibility polymorphism on the FTO gene located at rs9939609 results in decreased insulin secretion and increased ghrelin secretion leading to decreased glucose clearance and increased appetite, and is classed as a carbohydrate sensitivity susceptibility variant. In another example, the susceptibility polymorphism on the APOE gene located at rs429358 results in increased blood cholesterol and triglyceride levels and is classed as a lipid sensitivity susceptibility variant.

For each gene polymorphism selected, an odds-ratio risk calculation is performed using the mantel-haenszel test. The odds ratio refers to the odds of a susceptibility effect occurring in a group with the risk allele versus the odds of a susceptiility effect occurring in a group without the risk allele. Assuming risk allele=allele 1 (a1):

$$OR = \frac{( cases ( a 1 / a 2 ) )}{( contr . ( a 1 / a 2 ) )}$$

This OR is usually reported directly in the study that has analysed the SNP versus a susceptibility effect.

Given that the relative risk for the non-susceptibility risk allele, a2=1, the respective genotype relative risk is, where r=frequency of a1:
a1a1=r̂2
a1a2=r
a2a2=1

The odds ratio is used to calculate the odds of an event occurring (i.e. susceptibility) in one group to the odds of it occurring in another group. The relative odds of a particular outcome occurring for a particular genetic variant in the average population is calculated by using the frequency of the alleles for that variant in the average population. The allele frequencies of the SNP are obtained for the population to which the subject is a member of (i.e. Caucasian, African American, Han Chinese, etc.) from a database that maintains current records such as HAPMAP or directly from the scientific research studies. These frequencies are defined as p (susceptibility) and q, where:

p+q=1

The population frequencies of the 3 possible genotypes are then defined according to the Hardy Weinberg equilibrium:
a1a1=p̂2
a1a2=2pq
a2a2=q̂2

Under this assumption this enables the calculation of the average population sensitivity, defined as R, relative to the non-sensitivity genotype a2a2:

R=p̂2×r̂2+2pq×r+q

Finally, the sensitivity relative to the general population, defined RR, is calculated for each of the three possible genotypes:
a1a1: RR=r̂2/R
a1a2: RR=r/R
a2a2: RR=1/R

Where the sensitivity allele is not present the subsequent result would yield a RR less than 1 suggesting the genotype is not sensitive or normal against the sensitivity. Given that it is not possible to confirm such an interaction the result is instead assigned the neutral value 1 and no further modifications (see below) are applied.

Where the statistical power of studies used to calculate OR varies, a coefficient may be used. Given that OR values are obtained from publications with varying statistical strength it is important to discriminate between studies. Studies may be stratified according to power and concordance to derive a utility coefficient (UC).

For each concordant study published with a population:
<100: UC=1.05
101<500: UC=1.10
501<2000: UC=1.20
>2001: UC=1.25

Where more than one study exists, the product UC (PUC) may be derived by multiplying out all UC. Note that this rule is only applicable where the populations are common (i.e. all Caucasian). The PUC may be multiplied by each RR to derive a PRR.

Since the PRR may be being derived for more than one SNP per macronutrient susceptibility, each PRR may then multiplied to obtain an overall PRR for the respective sensitivity, defined as CSPRR for carbohydrate, and as LSPRR for lipid, i.e.:
CSPRR=product of all carbohydrate sensitivity PRR
LSPRR=product of all lipid sensitivity PRR

Note that the product rule stated above is only valid if the allele frequencies are common to the population being studied, i.e. if a Caucasian is being analysed then allele frequencies obtained from HAPMAP must be for Caucasian's for each SNP used in the scoring system of RR. Also note that the product rule assumes that each gene variant is randomly associating and there are no molecular or physiological interactions between variants.

The threshold for classifying sensitivity for an individual based on the genetic variation of multiple SNPs, unless otherwise stated, a multiplicative model for macronutrient sensitivity will be assumed.

The threshold for classifying sensitivity based on an aggregate product score is defined as a value of >1.00 (greater than 1.0), (or other threshold value deemed appropriate) for all variants included, where a minimum of 3 SNPs per sensitivity category are scored. Where the score is >1.00 (greater than 1.0), a subject is deemed sensitive for that macronutrient.

In another embodiment of a scoring system each SNP may be scored separately and where the PRR>=2 (or other threshold value deemed appropriate) it would constitute a positive mark against the sensitivity classification. Where 2 or more marks are obtained in a sensitivity classification the subject is deemed sensitive for that macronutrient.

Alternatively, or additionally, the subject's genetic profile may be used to recommend appropriate exercise and other lifestyle changes such as counselling to further increase the individual's satiation response and health benefits.

“Counselling” refers to the provision of advice, opinion, instruction and/or education, with the goal of directing the conduct of a subject. As used herein, such conduct relates to macronutrient sensitivity and in some instances compliance with a formulated diet and/or lifestyle.

As used herein, “exercise” refers to physical or psychological exertion for the sake of improvement, particularly in improving compliance with a formulated diet or for enhancing the satiety response achieved by compliance with a formulated diet. Physical exercise may include a psychological component.

Physical exercise may be aerobic or anaerobic, and the amount or ratio of each may be related to the genetic profile and macronutrient sensitivity of a subject.

The formulated diet or formulated food may include or be accompanied by a nutraceutical or a pharmaceutical. The subject's genetic profile may be used to recommend a nutraceutical or pharmaceutical. In one embodiment, a nutraceutical or a pharmaceutical is particularly suited to improving satiety. Alternatively, a nutraceutical or a pharmaceutical may improve a condition associated with satiety, for example obesity, increased circulating blood glucose, lipid and/or triglyceride levels.

A “nutraceutical” refers to a food or food-like substance which has health-giving or health-improving properties. A nutraceutical may include alpha-lipoic acid, cruciferous vegetable concentrate, glycine, idebenone, indole-3-carbinol, L-carnitine, lutein, lycopene, L-serine, N-acetyl-L-cysteine, quercetin dehydrate, glutamine, arginine and taurine.

A nutraceutical may include a botanical composition such as andrographis extract, artichoke extract, banaba leaf extract, bilberry leaf extract, cat's claw bark extract, curcumin root extract, cinnamon root extract, dandelion root extract, Epimedium grandiflorum extract, forskolin, garlic extract, Gingko biloba leaf extract, goldenseal root extract, green tea leaf extract, hawthorne extract, rosemary extract, schizandra berry, Scutellaria baicalensis, and silymarin.

A “pharmaceutical” refers to a substance, usually distinct from a food, introduced into the body for treating a condition or disease.

In one embodiment, appetite-suppressing drugs such as mazindol and derivatives of phenethylamine that act on noradrenergic neurotransmitters are included as part of the formulated diet, e.g., phenylpropanolamine, diethylpropion, phentermine, phendimetrazine, benzphetamine, amphetamine, methamphetamine, and phenmetrazine. Other appetite-suppressing drugs include sibutramine hydrochloric monohydrate, which acts as a monoamine (serotonin and norepinephrine) re-uptake inhibitor and affects the feeling of satiety (marketed under name Meridia, made by Abbot Laboratories), dexfenfluramine (Redux) and fenfluramine/phenteramine (Fen-phen), which act on the neurotransmitter serotonin.

A formulated diet that, in addition to inducing satiety, also includes measures to control appetite can further include other treatments for combating or preventing obesity. Substances useful for this purpose include, for example: hormones (e.g. catecholamines, glucagon, adrenocorticotropic hormone); clofibrate; halogenate; cinchocaine; chlorpromazine; drugs acting on serotonin neurotransmitters (e.g. fenfluramine, tryptophan, 5-hydroxytryptophan, fluoxetine, and sertraline); centrally active drugs (e.g. naloxone, neuropeptide-Y, galanin, corticotropin-releasing hormone, and cholecystokinin); a cholinergic agonist (e.g. pyridostigmine); a sphingolipid (e.g. lysosphingolipid or a derivative thereof); thermogenic drugs (e.g. thyroid hormone); ephedrine; beta-adrenergic agonists; drugs affecting the gastrointestinal tract (e.g. enzyme inhibitors such as tetrahydrolipostatin, indigestible food such as sucrose polyester, and inhibitors of gastric emptying such as threo-chlorocitric acid or its derivatives); beta-adrenergic agonists (e.g. isoproterenol and yohimbine); aminophylline to increase the beta-adrenergic-like effects of yohimbine, an alpha-2-adrenergic blocking drug (e.g. clonidine alone or in combination with a growth hormone releasing peptide); drugs that interfere with intestinal absorption (e.g. biguanides such as metformin and phenformin); bulk fillers (e.g. methylcellulose); metabolic blocking drugs (e.g. hydroxycitrate); progesterone; cholecystokinin agonists; small molecules that mimic ketoacids; agonists to corticotropin-releasing hormone; an ergot-related prolactin-inhibiting compound for reducing body fat stores; beta-3-agonists; bromocriptine; antagonists to opioid peptides; antagonists to neuropeptide Y; glucocorticoid receptor antagonists; growth hormone agonists; and combinations thereof.

Other pharmaceutical substances that can be included with the formulated diet or formulated food include, but are not limited to: rimonabant, which blocks the same pleasure receptor in the brain that responds to marijuana (marketed under the name Acomplia by Sanofi-Aventis, SA); intranasal PYY3-36 (PYY is a naturally occurring human hormone produced by specialized endocrine cells (L-cells) in the gut in proportion to the calorie content of a meal, PYY3-36 is a modified form of PYY and is studied by Nastech Pharmaceutical Company Inc.); Xenical, a molecule that attaches to lipases and blocks them from breaking down some of the lipid in the diet (Roche); energy consumption-increasing drugs; beta-3-adrenergic receptor agonists; and PPARgamma agonists.

The term “administer” or “administering” refers to delivery of a substance to a subject and ingestion of the substance by the subject. A substance may be self-delivered by the subject or may be delivered by another. Delivery may be simultaneous or sequential. Delivery may be achieved by incorporating the substance into the diet or may be achieved by separate ingestion.

“Treating” or “treatment” refers to both therapeutic treatment and prophylactic or preventative measures, wherein the aim is to prevent, ameliorate or lessen a health issue associated with macronutrient sensitivity.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

Diseases or disorders that may benefit from the present methods include, but are not limited to, metabolic syndrome, obesity, insulin resistance, glucose intolerance, dyslipidemia, non-alcoholic fatty liver disease, sleep apnoea, obesity-associated metabolic disorders such as osteoarthritis, type 2 diabetes, increased blood pressure, hypertension, stroke, heart disease, cardiovascular disease, osteoarthritis, unwanted weight gain (even where that weight gain is below the level of obesity) or unwanted body mass index, and excessive appetite resulting in unwanted weight gain.

It will be understood to persons skilled in the art of the invention that many modifications may be made without departing from the spirit and scope of the invention.

### EXAMPLES

### Example 1

Note panels can consist of any number of SNPs equal to greater than 3 per macronutrient susceptibility. For example, assume the usage of a

1) carbohydrate sensitivity panel comprised of:
FTO—rs9939609—surrogate marker based on metabolic dysfunction related to glucose metabolism
TCF7L2—rs7903146—surrogate marker based on metabolic dysfunction related to glucose metabolism
G6PC2—rs560887—surrogate marker based on metabolic dysfunction related to glucose metabolism

and a

2) lipid sensitivity panel comprised of:
APOE—rs4420638—surrogate marker based on metabolic dysfunction associated with lipid metabolism
PCSK9—rs11206510—surrogate marker based on metabolic dysfunction associated with lipid metabolism
APOB—rs693—surrogate marker based on metabolic dysfunction associated with lipid metabolism.

The subject being tested may be Caucasian and may have obtained the following results against the above panel for the forward strand (in square brackets are the allele frequencies for Caucasians for that SNP, followed by the published OR for the sensitivity allele for a Caucasian population):

Carbohydrate sensitivity panel
FTO—rs9939609 (AC)—[A:0.45 C:0.55|OR(A)=1.3]
TCF7L2—rs7903146 (CT)—[C:0.18 T:0.78|OR(C)=1.3]
G6PC2—rs560887 (GG)—[A:0.4 G:0.6|OR(A)=1.0]

Lipid sensitivity panel
APOE—rs429358 (AA)—[G:0.10 A:0.90|OR(G)=1.0]
PCSK9—rs11206510 (CC)—[C:0.77, T:0.23|OR (C)=1.0]
APOB—rs693 (GG)—[C:0.48 G:0.52|OR(A)=1.0]

The relative macronutrient sensitivity is calculated for each SNP according to the method mentioned in scoring methodology using the normal allele frequencies found in the general population.

For example, carbohydrate sensitivity panel
FTO—rs9939609 (AC)—1.3
TCF7L2—rs7903146 (CT)—1.2
G6PC2—rs560887 (GG)—0.97
Combined score 1.51

Lipid sensitivity panel
APOE—rs429358 (AA)—0.86
PCSK9—rs11206510 (CC)—1.0
APOB—rs693 (GG)—0.95
Combined score 0.817

Assuming that the statistical power across all studies does not vary and that there is no need to use a coefficient variable then if score for carbohydrate sensitivity=1.51 (i.e. >1.0) and the score for lipid sensitivity=0.817, the individual is, for the purposes of identifying macronutrient sensitivity, defined as carbohydrate sensitive.

### Example 2

Roughly 100 individuals were placed on the MyGene diet program. Individuals were assigned to a macronutrient sensitivity diet group based on genotype and monitored on a weekly basis for up to 6 months. Individuals assigned to a macronutrient sensitivity diet group based on genotype on average lost roughly 1 kg of fat mass per week whilst preserving lean mass until they reached their target goal weight whereby they were continually monitored thereafter.

A separate group of individuals that were double blinded and randomly assigned to a control diet that did not involve matching diet to genotype on average lost 4 kg in total over a 4 week period with most of this weight being lost from lean mass (2.4 kg) as opposed to fat mass (1.6 kg).

Importantly, individuals consuming a diet that was based on the individual's macronutrient sensitivity according to genotype consistently felt full whilst on the diet, reporting feelings of fullness (i.e. satiety) that lasted for, on average, 3.5-4 hours following meals. Conversely, those in the above mentioned control group, who consumed a diet that was not matched to genotype, on average started to feel hungry on average 2.5 hours following a meal.

Therefore, based on the above observations, a diet that is modified and given to an individual based on macronutrient sensitivity according to genotype, achieves benefits in terms of weight loss and lean mass preservation, as well as benefits associated with satiety and hunger control. As such, maintenance of a diet matched to an individuals macronutrient sensitivity according to genotype, in the long term is expected to reduce body fat, improve body composition by preserving lean mass, reduce the risk of weight regain following the diet by preserving lean mass and importantly, increase post-meal satiety and hunger control.

1-31. (canceled)

32. A method for determining a genetic predisposition of a subject to reduced satiety after consuming carbohydrate or lipid in excess of an optimal level for their genotype, comprising:
a) assaying a genetic sample from the subject for the presence of at least two polymorphisms associated with carbohydrate sensitivity and at least two polymorphisms associated with lipid sensitivity to obtain a polymorphism profile;
b) analysing the polymorphism profile to identify predisposition alleles;
c) calculating predisposition scores for carbohydrate sensitivity and lipid sensitivity from the identified predisposition alleles; and
d) classifying the subject's genetic predisposition to reduced satiety after consuming carbohydrate or lipid in excess of an optimal level for their genotype based on the predisposition scores.

33. The method of claim 32, wherein the at least two polymorphisms associated with carbohydrate sensitivity are selected from polymorphisms in genes selected from the group consisting of TCF7L2, FTO, KIR6.2 (KCJN11), PPARG, IGF2BP2, CDKN2B, SLC30A8, HHEX, CDKAL1, WFS1, NOTCH2, JAZF1, CDC123, G6PC2 and APOA5.

34. The method of claim 32, wherein the at least two polymorphisms associated with lipid sensitivity are selected from polymorphisms in genes selected from the group consisting of APOE, APOB, PSRC1, LDLR, CETP, LPL, PCSK9, FABP2 and LEPR.

35. The method of claim 32, wherein the polymorphisms are single nucleotide (SNPs) and wherein the SNPs associated with carbohydrate sensitivity are selected from the group consisting of SEQ ID NO: 1 (RS12255372), SEQ ID NO: 2 (RS7903146), SEQ ID NO: 7 (RS9939609), SEQ ID NO: 3 (RS5219), SEQ ID NO: 4 (RS1801282), SEQ ID NO: 5 (RS4402960), SEQ ID NO: 6 (RS10811661), SEQ ID NO: 8 (RS13266634), or SEQ ID NO: 9 (RS1111875), SEQ ID NO: 10 (RS7756992), SEQ ID NO: 11 (RS10010131), SEQ ID NO: 12 (RS10923931), SEQ ID NO: 13 (RS864745), SEQ ID NO: 14 (RS12779790), SEQ ID NO: 15 (RS560887), SEQ ID NO: 16 (RS12286037), and SEQ ID NO: 17 (RS662799).

36. The method of claim 32, wherein the polymorphisms are single nucleotide polymorphisms (SNPs) and wherein the SNPs associated with lipid sensitivity are selected from the group consisting of SEQ ID NO: 18 (RS4420638), SEQ ID NO: 19 (RS693), SEQ ID NO: 20 (RS754523), SEQ ID NO: 21 (RS599839), SEQ ID NO: 22 (RS6511720), SEQ ID NO: 23 (RS5882), or SEQ ID NO: 24 (RS708272), SEQ ID NO: 25 (RS320), SEQ ID NO: 26 (RS328), SEQ ID NO: 27 (RS11206510), SEQ ID NO: 28 (RS1799883), SEQ ID NO: 29 (RS8179183), and SEQ ID NO: 30 (RS1892534).

37. The method of claim 35, wherein the predisposition allele of SEQ ID NO: 1 (RS12255372) is T, SEQ ID NO: 2 (RS7903146) is T, SEQ ID NO: 7 (RS9939609) is A, SEQ ID NO: 3 (RS5219) is T, SEQ ID NO: 4 (RS1801282) is C, SEQ ID NO: 5 (RS4402960) is T, SEQ ID NO: 6 (RS10811661) is T, SEQ ID NO: 8 (RS13266634) is C, or SEQ ID NO: 9 (RS1111875) is G, SEQ ID NO: 10 (RS7756992) is G, SEQ ID NO: 11 (RS10010131) is G, SEQ ID NO: 12 (RS10923931) is T, SEQ ID NO: 13 (RS864745) is A, SEQ ID NO: 14 (RS12779790) is G, SEQ ID NO: 15 (RS560887) is G, SEQ ID NO: 16 (RS12286037) is T, or SEQ ID NO: 17 (RS662799) is G.

38. The method of claim 36, wherein the predisposition allele of SEQ ID NO: 18 (RS4420638) is G, SEQ ID NO: 19 (RS693) is T, SEQ ID NO: 20 (RS754523) is C, SEQ ID NO: 21 (RS599839) is A, SEQ ID NO: 22 (RS6511720) is G, SEQ ID NO: 23 (RS5882) is A, or SEQ ID NO: 24 (RS708272) is C, SEQ ID NO: 25 (RS320) is T, SEQ ID NO: 26 (RS328) is C, SEQ ID NO: 27 (RS11206510) is T, SEQ ID NO: 28 (RS1799883) is A, SEQ ID NO: 29 (RS8179183) is C or SEQ ID NO: 30 (RS1892534) is G.

39. The method of claim 32, comprising the further step of assaying the genetic sample to determine a haplogroup, optionally by assaying a mitochondrial polymorphism or a Y-chromosome polymorphism.

40. The method of claim 32, wherein the subject's genetic predisposition is classified as being non-sensitive, carbohydrate sensitive, lipid sensitive, or carbohydrate and lipid sensitive.

41. The method of claim 32, comprising the further step of formulating a diet for the subject based on their classification, including prescribing the diet or providing the diet as food.

42. The method of claim 32, comprising the further step of counselling the subject, and/or providing the subject with an exercise regimen, and/or to claim administering to the subject a nutraceutical or pharmaceutical substance, wherein the nutraceutical aids in normalising circulating glucose levels or circulating lipid and/or triglyceride levels.

43. A method for determining an appropriate diet to induce satiety in a subject, comprising:
a) determining the subject's genetic predisposition to reduced satiety after consuming carbohydrate or lipid in excess of an optimal level for their genotype by the method of claim 32; and
b) matching the subject's genetic predisposition to reduced satiety with a diet comprising appropriate levels of macronutrients for their genotype.

44. A kit for determining a genetic predisposition of a subject to reduced satiety after consuming carbohydrate or lipid in excess of an optimal level for their genotype, comprising a reagent for assaying a genetic sample from the subject for the presence of at least two polymorphisms associated with carbohydrate sensitivity and at least two polymorphisms associated with lipid sensitivity to obtain a polymorphism profile;
wherein the at least two polymorphisms associated with carbohydrate sensitivity are selected from polymorphisms in genes selected from the group consisting of TCF7L2, FTO, KIR6.2 (KCJN11), PPARG, IGF2BP2, CDKN2B, SLC30A8, HHEX, CDKAL1, WFS1, NOTCH2, JAZF1, CDC123, G6PC2 and APOA5; and
wherein the at least two polymorphisms associated with lipid sensitivity are selected from polymorphisms in genes selected from the group consisting of APOE, APOB, PSRC1, LDLR, CETP, LPL, PCSK9, FABP2 and LEPR.

45. The method of claim 32, wherein the genetic sample is a buccal sample.


SEQUENCE LISTING (30 sequences)

SEQ ID NO: 1 - DNA, 51 bp, Homo sapiens
  misc_feature (26)..(26): n is g or t
tgcccaggaatatccaggcaagaatnaccatattctgataattactcaggc

SEQ ID NO: 2 - DNA, 52 bp, Homo sapiens
  misc_feature (27)..(27): n is c or t
ttagagagctaagcactttttagatantatataatttaattgccgtatgagg

SEQ ID NO: 3 - DNA, 52 bp, Homo sapiens
  misc_feature (27)..(27): n is c or t
ccgctggcgggcacggtacctgggctnggcagggtcctctgccaggcgtgtc

SEQ ID NO: 4 - DNA, 52 bp, Homo sapiens
  misc_feature (27)..(27): n is c or g
aaactctgggagattctcctattgacncagaaagcgattccttcactgatac

SEQ ID NO: 5 - DNA, 52 bp, Homo sapiens
  misc_feature (27)..(27): n is g or t
cagtaaggtaggatggacagtagattnaagatactgattgtgtttgcaaaca

SEQ ID NO: 6 - DNA, 52 bp, Homo sapiens
  misc_feature (27)..(27): n is c or t
gcagctcacctccagctttagttttcncatgacagtaagtctattaccctcc

SEQ ID NO: 7 - DNA, 52 bp, Homo sapiens
  misc_feature (27)..(27): n is a or t
aggttccttgcgactgctgtgaatttngtgatgcacttggatagtctctgtt

SEQ ID NO: 8 - DNA, 52 bp, Homo sapiens
  misc_feature (27)..(27): n is c or t
gtgcttctttatcaacagcagccagcngggacagccaagtggttcggagaga

SEQ ID NO: 9 - DNA, 52 bp, Homo sapiens
  misc_feature (27)..(27): n is a or g
ctccgtaccatcaagtcatttcctctngacgtctgaacctgcactcagggtc

SEQ ID NO: 10 - DNA, 52 bp, Homo sapiens
  misc_feature (27)..(27): n is a or g
aatattcccccctgtattttagttttngatctacagttatgtagcaatgagc

SEQ ID NO: 11 - DNA, 52 bp, Homo sapiens
  misc_feature (27)..(27): n is a or g
gcacacaaggcctttgaccacatcctntccctcaggcatcacgtccgagaac

SEQ ID NO: 12 - DNA, 52 bp, Homo sapiens
  misc_feature (27)..(27): n is g or t
tcttgttgctccatcctctggcttcangctgaacaagtaagattatgggcac

SEQ ID NO: 13 - DNA, 52 bp, Homo sapiens
  misc_feature (27)..(27): n is a or g
catttcctacaaccattcaaaacattntaacagttcaaattatatttgagca

SEQ ID NO: 14 - DNA, 52 bp, Homo sapiens
  misc_feature (27)..(27): n is a or g
acccggacaatgttgggaattttttcntatttcttggccatttatatatctt

SEQ ID NO: 15 - DNA, 52 bp, Homo sapiens
  misc_feature (27)..(27): n is a or g
tctacgatggaagaatagatacaagcntaaaaagcaaagaaactggatcact

SEQ ID NO: 16 - DNA, 52 bp, Homo sapiens
  misc_feature (27)..(27): n is c or t
gactatagtacaatgtctttaccaaantggaagaccatagtgcagtcttcga

SEQ ID NO: 17 - DNA, 52 bp, Homo sapiens
  misc_feature (27)..(27): n is a or g
tgagccccaggaactggagcgaaagtnagatttgccccatgaggaaaagctg

SEQ ID NO: 18 - DNA, 52 bp, Homo sapiens
  misc_feature (27)..(27): n is a or g
caatgtcactatgctacacttttcctngtgtggtctacccgagatgaggggc

SEQ ID NO: 19 - DNA, 52 bp, Homo sapiens
  misc_feature (27)..(27): n is c or t
cacatgaaggccaaattccgagagacnctagaagatacacgagaccgaatgt

SEQ ID NO: 20 - DNA, 52 bp, Homo sapiens
  misc_feature (27)..(27): n is c or t
gtatttgcaaagtaggtgacaattgcntagtatccctaatatcaatacaaaa

SEQ ID NO: 21 - DNA, 52 bp, Homo sapiens
  misc_feature (27)..(27): n is a or g
aaagagaaagaaataggagcaggatcnacttccagatatacagagaatataa

SEQ ID NO: 22 - DNA, 52 bp, Homo sapiens
  misc_feature (27)..(27): n is g or t
ctcaccaatcaacctcttccttaaganaaaatgttaaggaagtcttaggcaa

SEQ ID NO: 23 - DNA, 52 bp, Homo sapiens
  misc_feature (27)..(27): n is a or g
ttgattggcagagcagctccgagtccntccagagcttcctgcagtcaatgat

SEQ ID NO: 24 - DNA, 52 bp, Homo sapiens
  misc_feature (27)..(27): n is c or t
acctggctcagatctgaaccctaactngaaccccagtgattctgggtctcag

SEQ ID NO: 25 - DNA, 52 bp, Homo sapiens
  misc_feature (27)..(27): n is g or t
acagagatcgctataggatttaaagcntttatactaaatgtgctgggatttt

SEQ ID NO: 26 - DNA, 52 bp, Homo sapiens
  misc_feature (27)..(27): n is c or g
ccatgacaagtctctgaataagaagtnaggctggtgagcattctgggctaaa

SEQ ID NO: 27 - DNA, 52 bp, Homo sapiens
  misc_feature (27)..(27): n is c or t
caaggatatagggaaaaccttgaaagngatgtctgtggtggccgtctttggc

SEQ ID NO: 28 - DNA, 52 bp, Homo sapiens
  misc_feature (27)..(27): n is a or g
ataaattcacagtcaaagaatcaagcncttttcgaaacattgaagttgtttt

SEQ ID NO: 29 - DNA, 52 bp, Homo sapiens
  misc_feature (27)..(27): n is c or g
ataattaatggagatactatgaaaaangagaaaaatgtcactttactttgga

SEQ ID NO: 30 - DNA, 52 bp, Homo sapiens
  misc_feature (27)..(27): n is a or g
ggaactttgtggttgcagtatgtcttnatccatcagcatattgtccaactcc